(19) United States
(12) Patent Application Publication
(10) Pub. No.: US 2025/0380938 A1
(43) Pub. Date: Dec. 18, 2025

(54) STERNUM SPREADER WITH A CROSSBAR HAVING AN ARCH WISE CURVED AND HORIZONTAL WISE CURVED LENGTH

(71) Applicant: Koros U.S.A., Inc., Moorpark, CA (US)

(72) Inventor: Tibor Koros, Westlake Village, CA (US)

(21) Appl. No.: 18/741,504

(22) Filed: Jun. 12, 2024

(51) Int. Cl. A61B 17/02; A61B 17/00

(52) U.S. Cl. CPC A61B 17/0206; A61B 2017/00407; A61B 2017/00473

(57) ABSTRACT

A sternum spreader surgical instrument includes a toothed cross bar having an end secured to a first end of a stationary arm forming a generally L-shaped section. A moveable arm has a first end coupled to the toothed cross bar in a generally perpendicular relation and has a housing with a rack and pinion structure to move the moveable arm along a length of the cross bar. Each arm is attached to a triangular shaped blade attachment having blades to engage the walls of a surgically opening. Rotating the rack and pinion structure spreads apart the arms to open the walls. The toothed crossbar has an arch wise and a horizontal wise curved length such that the arms can be spaced in, opened in and closed in both a generally arch wise and a horizontal wise, non-parallel relationships with their bottom surfaces and lengths both forming non-parallel angles.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the reproduction by anyone of the patent disclosure as it appears in the Patent and Trademark Office patent files or records, but otherwise reserves all copyright and trade dress rights whatsoever.

### BACKGROUND

### Field

This disclosure relates to a sternum spreader with a crossbar having an arch wise curved and horizontal wise curved length.

### Related Art

The heart is the muscular organ that pumps the blood. The heart is divided into four chambers-two ventricles referred to as the left and right ventricle, and two auricles referred to as the left and right auricle. The blood flow through these four chambers is controlled by four valves. Two of the valves, the mitral valve and the aortic valve can become diseased through injury or infection, or either can be genetically imperfect. These two valves can be surgically removed and replaced heart to replace an artificial heart valve.

Current heart surgery techniques include surgical machines or life support systems that are available in open heart surgery to allow the blood flow from the patient to be diverted to a heart-lung machine. The heart can be stopped while the surgeon performs surgery and repair upon the patient's heart.

It is desire to have a chest or sternum spreader used as a surgical instrument by a surgeon in an operating room to facilitate the surgeon in opening up the chest cavity of the patient and operating on the patient's heart. The spreader can be used during an operation to replace an aortic valve or mitral valve with an artificial heart valve. The sternum spreader can be used in bypass surgery, open heart, surgery, heart, transplant surgery, and the like. It is well known that a myriad of other heart repairs, replacements and surgeries can be assisted by a chest or sternum spreader.

### DESCRIPTION OF THE DRAWINGS

FIG. 1A is a top view of a sternum spreader.

FIG. 1B is a perspective view of the sternum spreader of FIG. 1A.

FIG. 1C is a cross-section view of the sternum spreader of FIG. 1A.

FIG. 1D is a top view of the teeth of a crossbar of a sternum spreader.

FIG. 1E is a cross-section view of a crossbar of a sternum spreader.

FIGS. 1F through 1I show various views of a sternum spreader having a vertically and horizontally bowed crossbar, which may be or be part of a sternum spreader herein.

FIG. 2 is a top view of a sternum spreader having triangular shaped blade attachments.

FIG. 3A is a top view of a sternum spreader.

FIG. 3B is a perspective view of the sternum spreader of FIG. 3A.

FIG. 4 is an operating environment/process for opening and closing a sternum spreader surgical instrument including a toothed crossbar having an arch wise curved and horizontal wise curved length.

FIG. 5 is an operating environment/process for configuring a sternum spreader.

Throughout this description, elements appearing in figures are assigned three-digit reference designators, where the most significant digit is the figure number and the two least significant digits are specific to the element. An element that is not described in conjunction with a figure may be presumed to have the same characteristics and function as a previously-described element having a reference designator with the same least significant digits.

### DETAILED DESCRIPTION

The systems, devices and methods described herein provide a sternum spreader with a crossbar having an arch wise curved and horizontal wise curved length. The sternum spreader can be described as a rib, sternal or chest expander or retractor. The crossbar can be described as having a length or as a rack (e.g., as in rack and pinion mechanics) with both a horizontal and vertical curve that are barrel shaped, bow shaped, angled, fan shaped, U shaped, C shaped or other curve shaped.

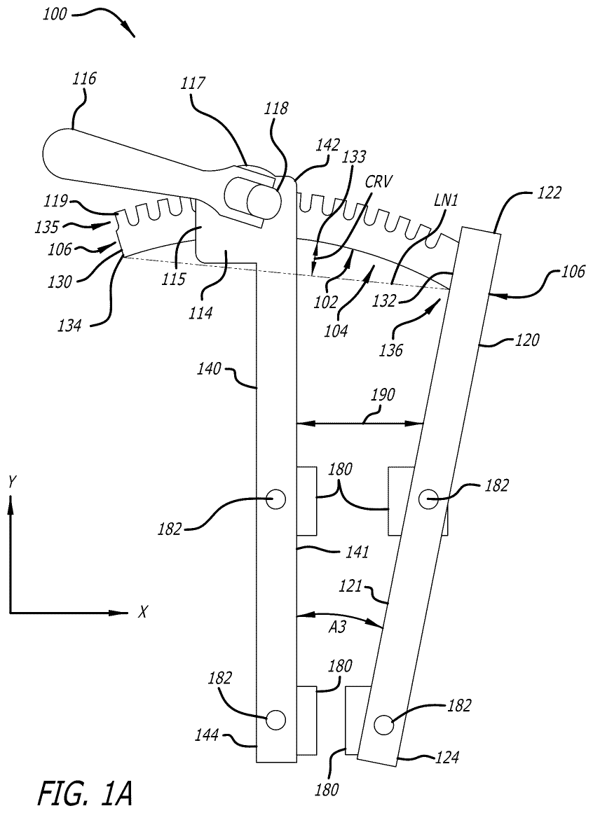

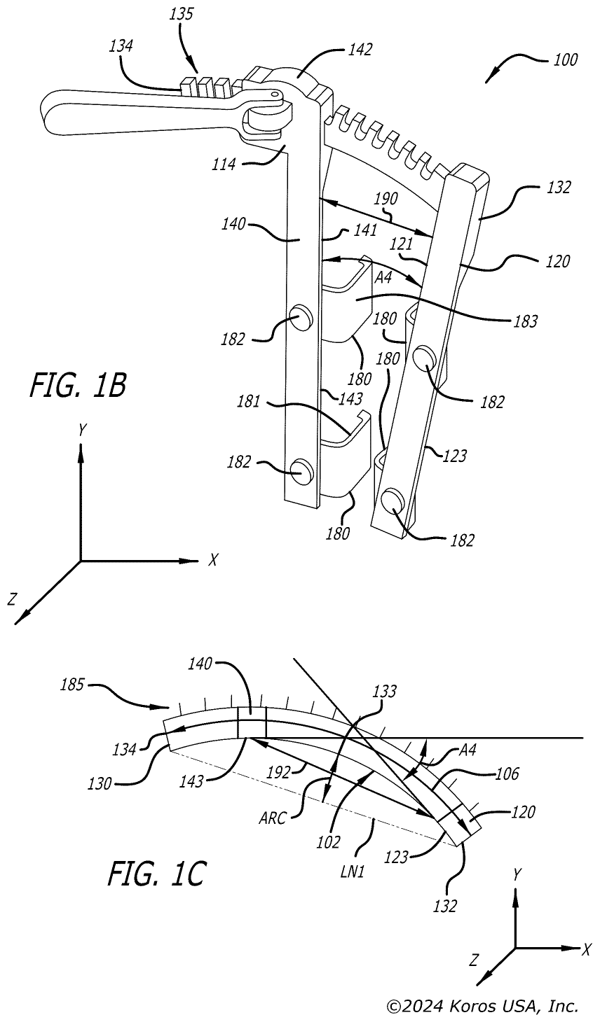

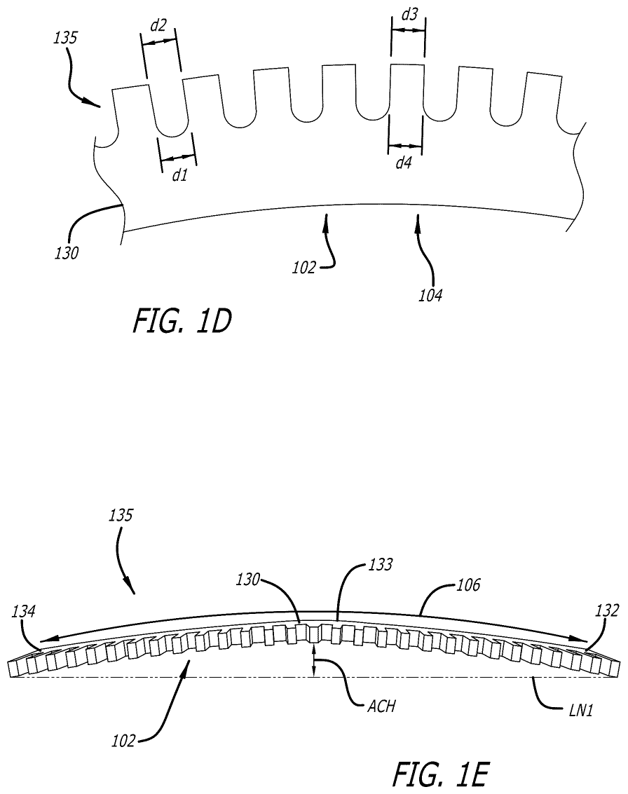

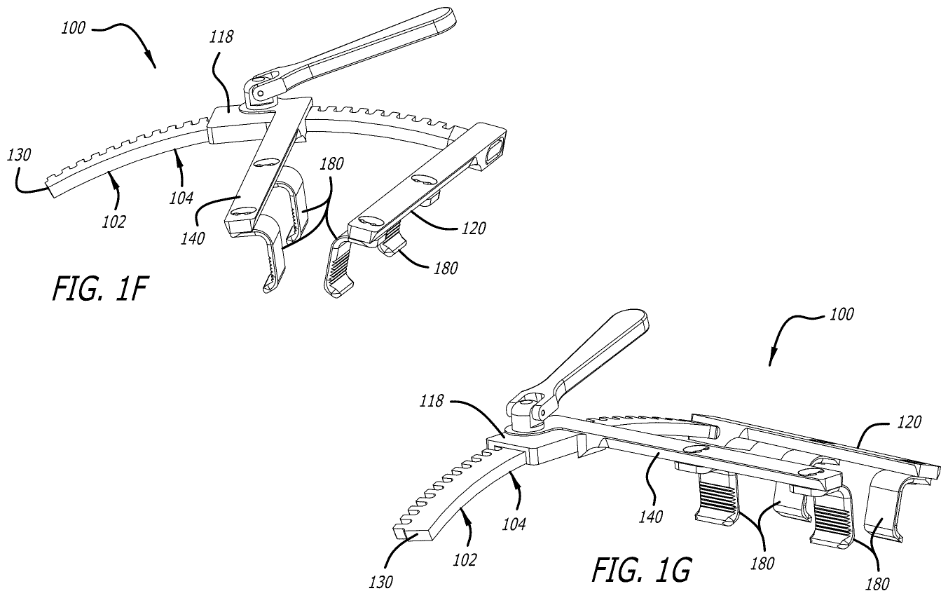

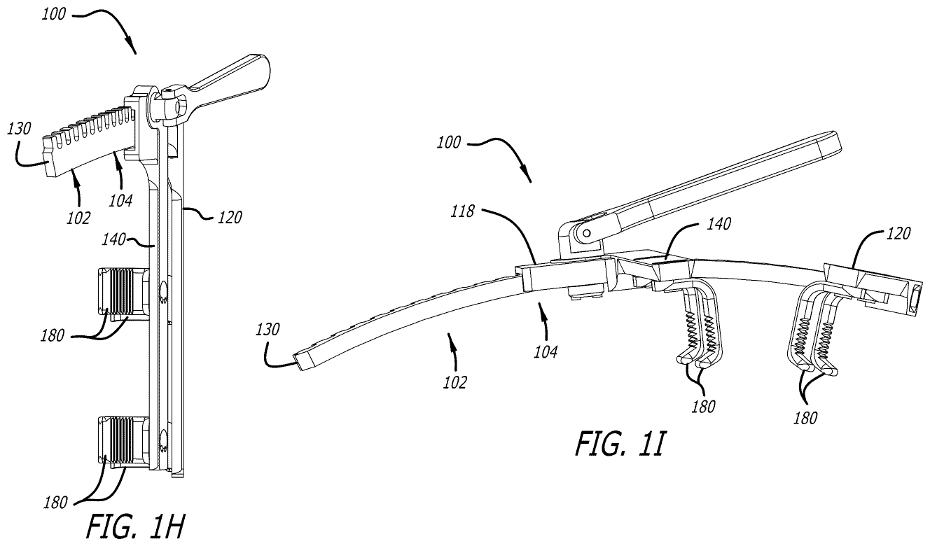

FIG. 1A is a top view of a sternum spreader 100 showing horizontally curved crossbar 130. FIG. 1B is a perspective view of a sternum spreader 100. FIG. 1C is a cross-section view of the sternum spreader 100 showing vertically bowed/arched crossbar 130. Spreader 100 can be used as a surgical instrument in open heart surgery to replace a damaged heart valve with an artificial one, in by-pass surgery, in aneurysm heart wall repair, in heart transplant surgery, and the like. Spreader 100 can be used in various other surgeries as appropriate.

Spreader 100 has a stationary arm 120 having a first end 122 proximal to a crossbar 130 and a second end 124 distal from the crossbar 130. Spreader 100 also has a moveable arm 140 having a first end 142 proximal to a crossbar 130 and a second end 144 distal from the crossbar 130.

Crossbar 130 is a toothed crossbar having a first end 132 and a second end 134, and a length 106 between ends 132 and 134. First end 132 (or near the first end) of crossbar 130 is secured or directly attached to the first end 122 of the stationary arm 120 in a generally perpendicular relation and forming a generally L-shaped section 136 between length 106 and the length 121 of arm 120.

The first end 142 of the movable arm 140 has a housing 114 with a slotted opening 115 which is attachable and shown attached to the toothed crossbar 130 and is laterally moveable along the length 106 of the crossbar. Part of or a location of length 106 of crossbar 130 is coupled to first end 142 of moveable arm 140 by housing 114 or rack and pinion structure 118 in a generally perpendicular relation with respect to length 106 and length 141 of arm 140.

Arms 120 and 140 are shown with locations 182 for removably attaching blades 180. Locations 182 may be anchor points, holes, slotted holes, or mounts of the arms at which the blades can be mounted by tungsten inert gas (TIG) welding or the like for proper use. A pin (at and also shown by location 182) may go through a hole in the top of the arms 120 and 140 at location 182 and also through a hole in blade 180 and be TIG welded at the base of the pin and under the bottom side of the blade 180 through hole at location 182. This may allow the blade to rotate freely about the pin and/or arm at location 182. In this case, the blades 180 are not removable. Locations 182 may be or have a blade swivel vertex, base or anchor that allows the blades 180 to swivel in the horizontal direction with respect to the lengths 121 and 141 of the arms. In some cases, the blades can be removably attached by being securely mounted for proper use and removed after use without damaging the arms or blades.

Locations 182 may be multiple positions along the stationary arm and the moveable arm for locating the blades. Although two locations 182 are shown on arms 120 and 140, additional locations 182 may be included with arms 120 and 140. Blades 180 have inner surfaces 181 towards the arms and outer surfaces 183 away from the arms.

Rack and pinion structure 118 in housing 114 is for moving the moveable arm 140 along the length 106 of the crossbar. Crossbar 130 may be a rack or linear gear 119 of a rack and pinion mechanical structure having pinion or circular gear 117 of structure 118 or housing 114. Structure 118 may include a circular gear 117 having pinion teeth and coupled to handle 116 for engaging a linear gear having rack teeth 135 along a rear surface of the length 106 of the crossbar 130 that is away from the length of arms 120 and 140. In FIG. 1C, teeth 135 are not shown because they are on the back side of crossbar 130 at locations 185.

The pinion structure 118 includes a handle 116 mounted on a housing 114 and attached to circular/pinion gear 117 for moving the arm 140 along length 106 with respect to arm 120. For example, gear 119 has teeth 135 that mechanically mesh with and move along teeth of gear 117 of pinion structure 118 when handle 116 is rotated, such as by a user of the spreader 100. The stationary arm 120 and the moveable arm 140 are oriented generally perpendicular to (that is, tangent or at a right angle with respect to) the length 106 of the crossbar 130 at a location where each arm is attached to the crossbar 130.

Length 106 has an arch wise curve 102 as well as a horizontal wise curve 104 along length 106. Curves 102 and 104 may be along all of length 106. In other cases, one or both of curves 102 and 104 may be along some but not all of length 106. Length 106 may be a length or a rack (e.g., as in rack and pinion mechanics) with both a horizontal curve 104 and vertical curve 102 that are barrel shaped, bow shaped, angled, fan shaped, U shaped, C shaped or other curve shaped; but not straight or linear in the direction of curve 104 or 102.

Crossbar 130 has the arch wise curved 102 and horizontal wise curved 104 length 106 such that the stationary arm 120 and the moveable arm 140 are capable of being: (1) spaced in both generally arch wise, non-parallel relationship 190 with their bottom surfaces at angle A4; and generally horizontal wise, non-parallel relationship 192 with their first ends 122 and 142 farther apart than their second ends 124 and 144; and (2) opened and closed in both the generally arch wise, non-parallel relationship 190 and horizontal wise, non-parallel relationship 192 to each other by rotating the rack and pinion structure 118 or handle 116 in the housing 114 of the moveable arm 140.

In some cases, the moveable arm 140 and stationary arm 120 are or are capable of being: (1) spaced in with their first ends and their second ends separated by the blade attachments, and (2) opened and closed by rotating the rack and pinion structure in the housing of the moveable arm.

Non-parallel relationships 190 and 192 may each be a non-zero angled separation between the inner surfaces or lengths 121 and 141 of arms 120 and 140. They may also cause a non-zero angled separation between the inner surfaces 181 of the blades 180, such as where top part 262 of the blades is wider than bottom part 264 of the blades in the Z or vertical direction.

Length 106 may be between 2 and 14 inches from end 132 to end 134 of the crossbar 130. Length 106 may be between 2 and 12 inches or between 2 and 11 inches. In some cases, length 106 may be between 6 and 12 inches, such as for an adult patient. Length 106 may be between 2 and 5 inches, such as for a child or pediatric patient. FIG. 1C shows an arch distance ACH in the Z direction between a line LN1 connecting the ends 132 and 134 of the crossbar 130 and the midpoint 133 of the crossbar 130. Arch wise curve 102 of the length 106 has (e.g., a first curve distance) an arch distance ACH of between 0.25 to 3 inches at middle 133 of crossbar length 106. In some cases, ACH is between 0.5 and 2 inches. Arch wise curve 102 is or is part of relationship 192.

FIG. 1A shows a curve distance CRV in the Y direction between line LN1 connecting the ends 132 and 134 of the crossbar 130 and the midpoint 133 of the crossbar 130. Horizontal wise curve 104 of the length 106 has (e.g., a second curve distance) a curve distance CRV at the center point of length 106 of between 0.25 to 3 inches at middle 133 of crossbar length 106. In some cases, CRV is between 0.5 and 2 inches. Horizontal wise curve 104 is or is part of relationship 190.

In some cases, the toothed crossbar 130 has two curved axes 102 and 104 with one curved/arched axis 104 in the X, Y-horizontal direction; and another curved/arched axis 102 in the Z-vertical direction. In other cases, it has only one curve 102 or 104, which is distinct from having both curves 102 and 104.

Structure 118 and arm 140 can be locked in any position along length 106 by mechanism in structure 118.

FIG. 1D is a top view of teeth 135 of crossbar 130 of a sternum spreader, which may be a spreader herein. FIG. 1D shows teeth 135 having a larger or wider spacing d2 between the distal end of the teeth away from the crossbar 130 than spacing d1 between the proximal end of the teeth closer to the crossbar 130. FIG. 1D also shows teeth 135 having a larger or wider distal end d3 away from the crossbar 130 than proximal end d4 towards the crossbar 130. Larger or wider distal spacing d2 and/or larger or wider distal end d3 may be due to having curve 102 and/or curve 104.

In some cases, teeth 135 have both, larger or wider distal spacing d2 and larger or wider distal end d3. In other cases, teeth 135 have only larger or wider distal spacing d2 or larger or wider distal end d3, but not both.

FIG. 1E is a cross-section back view of a vertically bowed crossbar 130 of a sternum spreader, which may be a spreader herein. FIG. 1E shows teeth 135 along length 106. FIG. 1E shows an arch distance ACH between a line connecting the ends 132 and 134 of the crossbar 130 and the midpoint 133 of the crossbar 130.

FIGS. 1F through 1I show various views of a sternum spreader 100 having a vertically bowed crossbar 130, which may be or be part of a spreader herein.

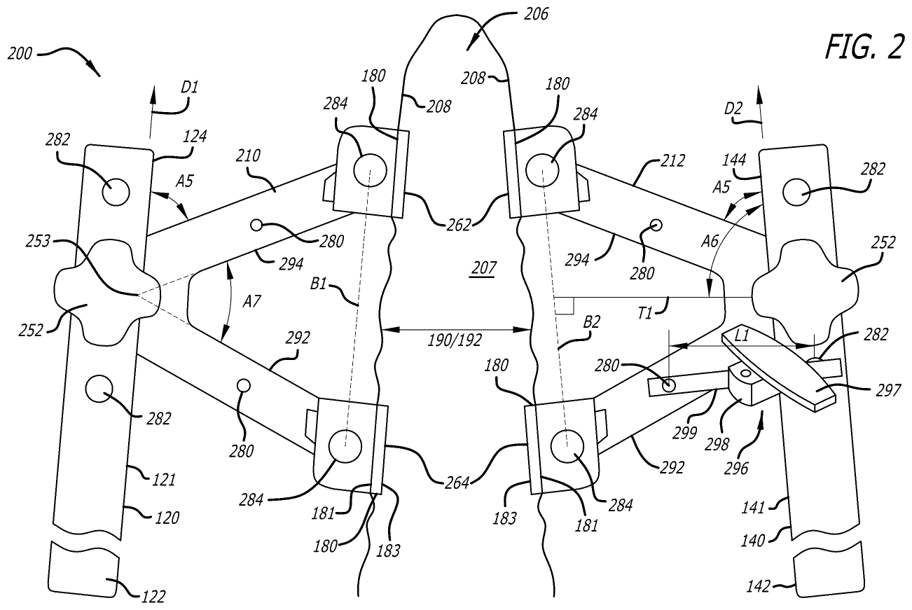

FIG. 2 is a top view of a sternum spreader 200 having triangular shaped blade attachments 210 and 212 removably attached to arms 120 and 140, respectively. Spreader 200 can be used for surgeries similar to descriptions for using spreader 100. Spreader 200 may be similar to and include structures of spreader 100 but may include the blade attachments 210 and 212 removably attached at locations 282 of the arms and having the blades 180 attached to the blade attachments instead of attached directly to locations 182 of the arms.

FIG. 2 shows that each of the moveable arm 140 and the stationary arm 120 are attached to or have a triangular shaped blade attachment 212 and 210 having a pair of blades 180 connected or attached to and configured to have inner surfaces 181 of the blades 180 engage the walls 204 of a surgically cut opening 206 on a patient. Blades 180 may extend below each of the blade attachments such that inner surfaces 181 of the blades 180 can engage the walls 204 of a surgically cut opening 206 on a patient. The blades may not be removable. In other cases, the blades may be removably attached.

The movable arm 140 can be extended away from the stationary arm 120 by rotating the handle 116 or pinion circular gear 117 of the rack and pinion structure 118 to spread apart and hold open the walls 208 of the opening 206 thereby exposing a body or chest cavity 207 for allowing a surgeon to operate in the cavity 207, such as on a heart or other cardiovascular structures.

The stationary arm 120 and the moveable arm 140 may be arch wise (e.g., in the up and down, vertical or Z direction) in a non-parallel relationship 192 with each other such that the top surfaces and/or bottom surfaces 123 and 143 of the stationary arm 120 and the moveable arm 140 are not parallel. There is an arch wise angle A4 (see FIGS. 1B and 1C) between the bottom surfaces 123 and 143 that increases from approximately 0 or 2 degrees to 25 degrees as the movable arm 140 is extended away from the stationary arm 120. In some cases, angle A4 extends from 0 to 20 degrees. Angel A4 may also exist between the inner surfaces 181 of the blades coupled to arm 120 as compared to those coupled to arm 140.

The stationary arm 120 and the moveable arm 140 may be horizontal wise (e.g., in the left and right, horizontal or X/Y direction) in a non-parallel relationship 190 with each other such that the lengths 121 and 141 of the arms are not parallel. There is a horizontal wise angle A3 (see FIG. 1A) between the lengths 121 and 141 of the arms (and a vertex found by extending from those lengths) that increases from approximately 0 or 2 degrees to approximately 45 degrees as the movable arm 140 is extended away from the stationary arm 120. In some cases, angle A3 extends from 0 to 30 degrees. Both angles A3 and A4 can be selected by rotating rack and pinion structure 118 to move arm 140 towards and away from arm 120.

Each triangular shaped blade attachment 210 and 212 may be an assembly, structure, housing or frame that swivels in the horizontal direction (X and Y) with respect to the lengths 121 and 141.

In FIG. 2, arms 120 and 140 are shown with locations 282 for attaching triangular shaped blade attachments 210 and 212. Locations 282 may be anchor points, holes, slotted holes or mounts of the arms at which the blade attachments can be removably attached by being securely mounted for proper use and removed after use without damaging the arms, blades or blade attachments. Locations 282 may be locations 182. The blade attachments 210 and 212 may be removable from mounts 282 so the blade attachments can be interchanged with other blade attachments, such as ones having different angles A7, of arms 292 and 294, vertical heights between fasteners 252 and locations 284, and/or thicknesses. Locations 282 may be multiple positions along the lengths of the stationary arm and the moveable arm for locating the blade attachments.

Each of triangular shaped blade attachments 210 and 212 include a proximal fastener 252 attached to one of locations 282 of the stationary and moveable arms; and each have two distal blade mounts 284 for mounting a first and second blade 180. Mounts 284 may be similar to locations 182 but on the blade attachments instead of on the arms. For example, a pin (at and also shown by location 284) may go through a hole in the top of the arms 292 and 294 at location 284 and also through a hole in blade 180 and be TIG welded at the base of the pin and under the bottom side of the blade 180 through hole at location 284. This may allow the blade to rotate freely about the pin and/or arm at location 284. In this case, the blades 180 are not removable. Locations 284 may be or have a blade swivel vertex, base or anchor that allows the blades 180 to swivel in the horizontal direction with respect to the lengths of the arms. In some cases, the blades can be removably attached by being securely mounted for proper use and removed after use without damaging the arms or blades.

Proximal fasteners 252 may be removably attached to the arms at locations 282. In other cases, they may be permanently attached to the arms at the locations. Proximal fasteners 252 may be or have a blade attachment swivel vertex, base or anchor that allows the blade attachments 210 and 212 to swivel in the horizontal direction with respect to the lengths 121 and 141 of the arms, respectively.

Proximal fasteners 252 swivel or pivot with respect to the lengths 121 and 141 between the first and second end of the arms 130 and 140 so the triangular shaped blade attachments 210 and 212 swivel in the horizontal direction at an angle A5 that changes between the lengths 121 and 141 of the arms and lengths of arms 294 of each blade attachment. Each triangular shaped blade attachment 210 and 212 swivels or pivots in the horizontal direction (X and Y) with respect to the lengths 121 and 141 to form angle A6 between the lengths 121 and 141 and a tangent Tl of lines B1 and B2, respectively.

Each of triangular shaped blade attachments 210 and 212 has blade attachment arms 292 and 294 extending from the fasteners 252 to locations 284. The blade attachment arms 292 and 294 may each be between 3 and 5 inches in length from the fasteners 252 or location 282 to locations 284. In some cases, the arms have equal lengths. In other cases, they do not have equal lengths.

Distal blade mounts 284 are for mounting first and second blades 180. Blades may be non-removably mounted on the arms 292 and 294. Distal blade mounts 284 may be or have a blade swivel vertex, base or anchor that allows the blades 180 to swivel in the horizontal direction with respect to the lengths of the arms 292 and 294.

The location of the fasteners 252 and mounts 284 form a triangular shape in the horizontal direction. The shape may be horizontally flat. The shape may have vertical steps such as along arms 292 and 294. An angle A7 is at a vertex 253 at the location of the fasteners 252 between rays extending from that vertex to mounts 284. The angel A7 may be between 10 and 120 degrees. It may be between 25 and 65 degrees. In some cases, it is between 40 and 50 degrees. It may be 45 degrees.

Distal blade mounts 284 may swivel or pivot with respect to the blade attachments so an angle (not shown) changes between a flat inner 181 surface of the blade and lines B1 and B2 connecting the two blade mounts 284 of each blade attachment. Inner surfaces 181 and or outer surfaces 183 of the blades may be perpendicular to top surfaces, bottom surfaces or lengths of the blade attachment arms 292 and 294.

The fasteners 252 may be removable from locations 282 of the arms 120 and 140 so the blade attachments can be interchanged with other triangle attachment attachments, such as ones having different attachment arm lengths, arm angles, and vertical locations of mounts 284 with respect to locations 282. Mounts 284 may be removable mounts so blades 180 can be interchanged with other blades, such as ones having different lengths, heights or depths, thicknesses, shapes and/or curves.

FIG. 2 also shows an attachment angle adjuster 296 attached to and between the arm 140 and an arm 292 of the blade attachment 212. Adjuster 296 extends from one of the locations 282 of arm 140 to an angle adjuster mounting location 280 of the blade attachment 212.

Adjuster 296 has an extendable length L1 between locations 282 and 280 that may be between 0.25 and 6 inches, and controls angle A5 of between 0 and 45 degrees between a length of arm 292 and length 141 of arm 140. Length L1 may be between 1 and 4 inches. Angle A5 may be between 5 and 30 degrees. It may be between 0 and 25 degrees. Adjuster 296 also controls angle A6 by between 10, 15, 20 or 30 degrees from a 45 degree angle.

Length L1 and angle A5 can be selected by rotating rack and pinion structure 298 to extended and retract rack 299 extending through structure 298. The rotating may be rotating handle 297 or a pinion circular gear of structure 298 to move linear gears of rack 299 with respect to the pinion gear.

In addition to adjuster 296 shown between a lower location 282 of the arm 140 and a lower location 280 of lower arm 292 of the blade attachment 212, another adjuster 296 can also be located between the lower location 282 of the arm 120 and the lower location 280 of arm 292 of the blade attachment 210.

Although adjuster 296 is shown between a lower location 282 of the arm 140 and a lower location 280 of lower arm 292 of the blade attachment 212, adjuster 296 can instead be located between the upper location 282 of the arm 140 and the upper location 280 of arm 294 of the blade attachment 212. In other cases, an adjuster 296 is located at both the upper and lower locations 282 and 280 of arm 140 and blade attachment 212.

Also, an adjuster 296 can be located between a lower location 282 of the arm 120 and a lower location 280 of lower arm 292 of the blade attachment 210, or between the upper location 282 of the arm 120 and the upper location 280 of arm 294 of the blade attachment 210. In other cases, adjuster 296 is located at both the upper and lower locations 282 and 280 of arm 120 and blade attachment 210.

It is considered that adjuster 296 can be located between any one, two, three or four of the lower locations and/or upper locations 282 and 280 of the arm 120 and/or 140.

Thus, locations 282 and 280 are for attaching to adjuster 296. Locations 282 and 280 may be anchor points, holes, slotted holes or mounts of the arms at which the adjuster 296 can be removably attached by being securely mounted for proper use and removed after use without damaging the spreader arms, blade attachment arms or blade attachments. One end of rack 299 may be attached to location 280, while structure 298 is attached to location 282 as shown, and another end of rack 299 extends through structure 298 with an adjustable length selected by rotation structure 298. This rotation selects length L1 as the total length of rack 299 less the length of the rack 299 extending through the structure 298.

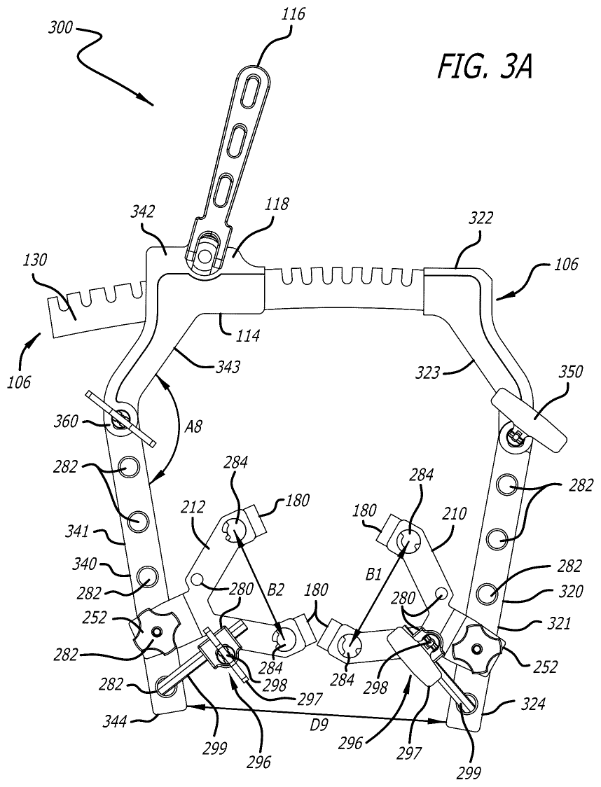

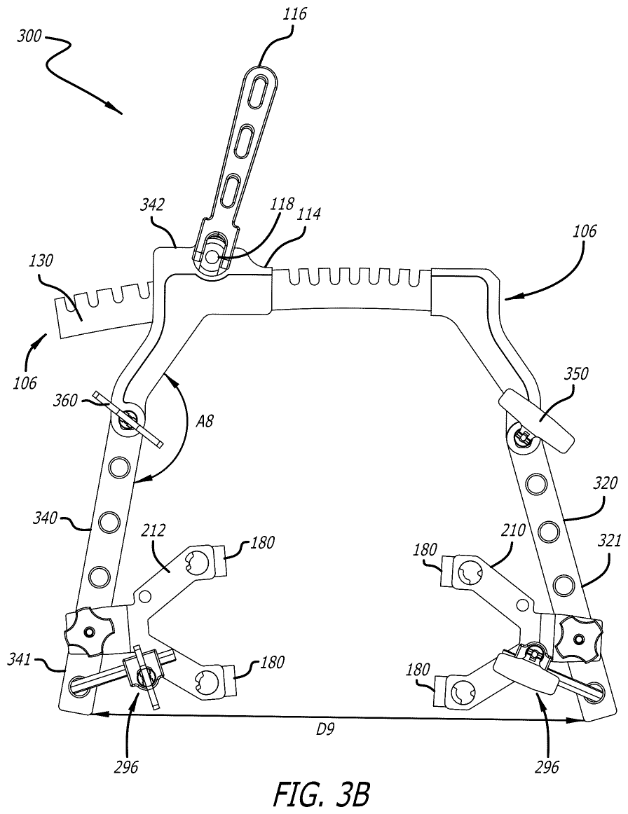

FIG. 3A is a top view of a sternum spreader 300. FIG. 3B is a perspective view of the sternum spreader 300. Spreader 300 may be part of or include any structure of spreaders 100 and/or 200. Spreader 300 may be similar to and include structures of spreader 100 or 200 but may include the arm angle adjustors 350 and 360 as part of arms 120 and 140 respectively to create arms 320 and 340 as shown.

The stationary arm 320 and moveable arm 340 include arm angle adjustors 350 and 360 to change a distance D9 between distal ends 324 and 344 of the arms 320 and 340. The arm angle adjustors 350 and 360 may also change an angle A8 with a vertex at the arm angle adjustor and between proximal lengths 323 and 343 of the arms and distal lengths 321 and 341 of the arms 320 and 340, respectively. Distance D9 may be between 1 and 7 inches. It may be between 3 and 6 inches.

Angle A8 may be between 140 and 230 degrees. It may be between 160 and 200 degrees. It may be between 160 and 180 degrees.

Distance D9 and angle A8 can be selected by rotating rack and pinion structures of arm angle adjustors 350 and 360 to change angle A8.

Instead of rack and pinion structures described herein, other location or length adjustments may be used such as a mechanical friction slide, a screw and nut structure, or a threaded receiver and bolt structure.

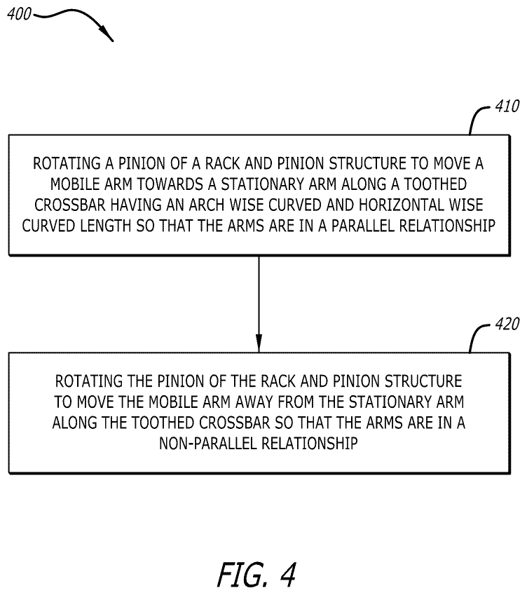

FIG. 4 is an operating environment/process 400 for opening and closing a sternum spreader surgical instrument including a toothed crossbar having an arch wise curved and horizontal wise curved length. Process 400 may be for opening and closing a sternum spreaders described herein, such as spreader 100, 200 and/or 300. Process 400 begins at step 410 and ends at step 420 but may be repeated such as by returning to step 410 after step 420. Process 400 may begin at step 420 and end at step 410.

At step 410, pinion gear 117 of a rack and pinion structure 118 is rotated to move a mobile arm 140 towards a stationary arm 120. Rotating at step 410 includes moving the mobile arm 140 along a toothed crossbar 130 having an arch wise curved 102 and horizontal wise curved 104 length 106 so that the arms 120 and 140 are in a parallel relationship (not shown) with respect to their lengths 121 and 140. In some cases, rotating at step 410 includes rotating so that blades 180 or parts of blade attachments 210 and 212 of the arms 120 and 140 are touching.

After step 410 the blades 180 may be placed or inserted into an incision in the chest of a patient so that the blades are capable of or configured to open, split or “crack” the chest of the surgical patient, such as for heart surgery, bypass or replacement. In other cases, at step 410, no patient is present and the spreader is being exercised, maintenance or tested.

Then, at step 420, pinion gear 117 of a rack and pinion structure 118 is rotated to move a mobile arm 140 away from stationary arm 120. Rotating at step 420 includes moving the mobile arm 140 along a toothed crossbar 130 having an arch wise curved 102 and horizontal wise curved 104 length 106 so that the arms 120 and 140 are in a non-parallel relationship (e.g., see FIGS. 1A-3B) with respect to their lengths 121 and 140, and with respect to their bottom surfaces 132 and 143. In some cases, rotating at step 420 includes rotating so that blades 180 or parts of blade attachments 210 and 212 of the arms 120 and 140 are not touching; or so that the blades are capable of or configured to open, split or “crack” the chest of a surgical patient, such as including by moving bone segments and/or ribs on the patient's left side away from those on the patient's right side. Step 420 may include forming a non-parallel relationship 190 between a more narrow top part 262 of the spreader and opening 206 towards the patient's neck, and a wider bottom part 264 of the spreader creating a wider part of the opening towards the patient's feet. Step 420 may include forming a non-parallel relationship 192 between a more narrow top part 262 of the spreader and opening 206 towards the patient's neck, and a wider bottom part 264 of the spreader creating a wider part of the opening towards the top of the patient's chest or cavity 207 as compared to deeper in the chest or cavity.

After step 420, step 410 may be repeated so that the blades are capable of or configured to close the chest of the surgical patient, such as including by moving bone segments and/or ribs on the patient's left side towards or touching those on the patient's right side.

In other cases, a patient is not present and the spreader is being exercised, maintenance or tested to have more top part 262 more narrow than bottom part 264

In some cases, at step 410 and/or 420 each of the mobile arm 140 and the stationary arm 120 are attached to a triangular blade attachment 212 and 210, and each triangular blade attachment is attached to two blades 180, such as shown in FIGS. 2-3B. In other cases, the blades 180 can be directly attached to locations 182 of the arms, such as shown in FIGS. 1A-1B.

At step 410, handle 116 can be manually rotated, e.g. clockwise, to move arm 140 in a first non-linear direction relative to arm 120; and at step 420, handle 116 can be manually rotated oppositely, e.g., counter clockwise, to move arm 140 in a first non-linear direction relative to arm 120 in an opposite direction. To facilitate a user's or surgeon's ability to precisely incrementally move the arm 140 (and blades attached to that arm) relative to the arm 120, the rack and pinion assembly 114 preferably defines discrete detent positions which provide discrete step tactile feedback to the user as the handle 116 is rotated.

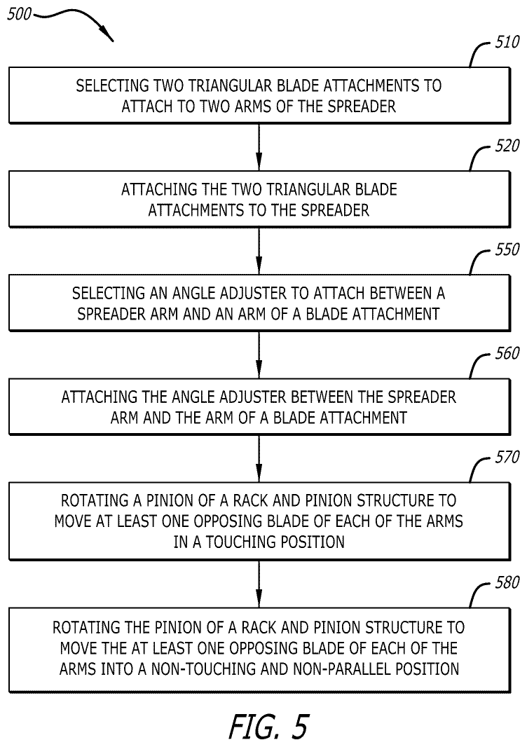

FIG. 5 is an operating environment/process for configuring a sternum spreader. Process 500 may be for configuring a sternum spreader described herein, such as spreader 100, 200 and/or 300. Process 500 begins at step 510 and ends at step 580 but may be repeated such as by returning to step 510, 550 or step 570 after step 580. Process 500 may only include one pair of steps 510/520 or 550/560. It may only include two of those pair. It may only include all three pair.

At step 510, two triangular blade attachments 210 and 212 are selected to attach to two arms 120 and 140 of the spreader.

At step 520, the selected two triangular blade attachments 210 and 212 are attached to the two arms 120 and 140 of the spreader.

At step 520, the blade attachments 210 and 212 may be removably attached to mounts 282 so the blade attachments can be interchanged with other blade attachments, such as ones having different angles A7, of arms 292 and 294, vertical heights between fasteners 252 and locations 284, and/or thicknesses. In some cases, after step 520, steps 510-520 are repeated to make such an interchange.

At step 550, an angle adjuster 296 is selected to attach between a spreader arm 120 or 140 and an arm 292 or 294 of a blade attachment 210 or 212.

At step 560, the selected angle adjuster 296 is attached between the spreader arm 120 or 140 and the arm 292 or 294 of the blade attachment 210 or 212.

Steps 550 and 560 may include selecting and attaching the angle adjuster 296 to the selected arm 292 or 294 and blade attachment 210 or 212.

At step 560 the angle adjuster 296 may be removably attached to selected arm 292 or 294 and blade attachment 210 or 212 so the angle adjuster 296 can be interchanged with other angle adjusters, such as ones having different lengths, heights, thicknesses, shapes and/or curves. In some cases, after step 560, steps 550-560 are repeated to make such an interchange.

At step 570, rack and pinion structure 118 is rotated to move a mobile arm 140 towards a stationary arm 120 so that at least one opposing blade 180 of each of the arms 120 and 140 (or of blade attachments 210 and 212) is in a touching position. This may apply to spreader 100, 200 or 300. For example, rotating at step 570 may include moving the mobile arm 140 along a toothed crossbar 130 having an arch wise curved 102 and horizontal wise curved 104 length 106 so that the arms 120 and 140 are in a parallel relationship (not shown) with respect to their lengths 121 and 140. In some cases, rotating at step 510 includes rotating so that blades 180 or parts of blade attachments 210 and 212 of the arms 120 and 140 are touching.

After step 570 the blades 180 may be placed or inserted into an incision in the chest of a patient so that the blades are capable of or configured to open, split or “crack” the chest of the surgical patient. In other cases, at step 510, no patient is present and the spreader is being exercised, maintenance or tested.

At step 580, rack and pinion structure 118 is rotated to move a mobile arm 140 away from stationary arm 120 so that at least one opposing blade 180 of each of the arms 120 and 140 (or of blade attachments 210 and 212) is in a non-touching and non-parallel position.

Rotating at step 580 may include moving the mobile arm 140 along a toothed crossbar 130 having an arch wise curved 102 and horizontal wise curved 104 length 106 so that the arms 120 and 140 are in both non-parallel relationship (e.g., see FIGS. 1A-3B) 190 with respect to their lengths 121 and 140, and relationship 192 with respect to their bottom surfaces 132 and 143.

In some cases, rotating at step 580 includes rotating so that blades 180 or parts of blade attachments 210 and 212 of the arms 120 and 140 are not touching; or so that the blades are capable of or configured to open, split or “crack” the chest of a surgical patient, such as including by moving bone segments and/or ribs on the patient's left side away from those on the patient's right side. Step 580 may include forming a non-parallel relationships 190 and 192 between a more narrow top part 262 of the spreader and opening 206 towards the patient's neck, and a wider bottom part 264 of the spreader creating a wider part of the opening towards the patient's feet and towards the upper part of the chest.

After step 580, step 570 may be repeated so that the blades are capable of or configured to close the chest of the surgical patient, such as including by moving bone segments and/or ribs on the patient's left side towards or touching those on the patient's right side.

In other cases, at steps 570-580 a patient is not present and the spreader is being exercised, maintenance or tested to have more top part 262 more narrow than bottom part 264

At step 570, handle 116 can be manually rotated, e.g. clockwise, to move arm 140 in a first non-linear direction relative to arm 120; and at step 580, handle 116 can be manually rotated oppositely, e.g., counter clockwise, to move arm 140 in a first non-linear direction relative to arm 120 in an opposite direction. To facilitate a user's or surgeon's ability to precisely incrementally move the arm 140 (and blades attached to that arm) relative to the arm 120, the rack and pinion assembly 114 preferably defines discrete detent positions which provide discrete step tactile feedback to the user as the handle 116 is rotated.

Spreaders or parts of spreaders herein may be manufactured of or including metal or alloy such as steel, stainless steel, aluminum, brass, titanium, tungsten, and/or other materials know for forming medical instruments or sternum spreaders. They may be or include a hard oxide, carbide, resin, epoxy, and/or porcelain. They may manufactured and/or shaped using forging, molding, cutting, laser cutting, water cutting and/or as known for forming medical instruments or sternum spreaders.

Having a device with; configuring, selecting and/or using a device with crossbar 130, blade attachments 210 and 212, and/or attachment angle adjuster 296 allows the blades 180 to be placed or inserted into an incision in the chest of a patient so that the blades are capable of or configured to open, split or “crack” the chest of the surgical patient with a more narrow top part 262 of the spreader creating a more narrow part of the opening 206 towards the patient's neck, and a wider bottom part 264 of the spreader creating a wider part of the opening towards the patient's feet. In other cases, a patient is not present and the spreader is being exercised, maintenance or tested to have more top part 262 more narrow than bottom part 264.

Having top part 262 wider than bottom part 264 provides the unexpected benefit of post surgical reduction patient pain and healing time. It may also provide benefits during the surgery such as reducing the risk that the chest will tear above the top of the incision; which may increase post surgery pain and healing time. It may avoid splitting the ribcage above 85 percent of the sternum or sternum body; to or through the manubrium; and/or through or past the top or first ribs of a patient. It is considered that the sternum spreaders, crossbar 130, blade attachments 210 and 212, and/or attachment angle adjuster 296 herein can be used with other removable and moveable attachments (e.g., to mount at various locations 182, 282 and/or 284) such as for mitral, aortic or tricuspid valve repair or replacement. They may also be used to provide solid adjustable platforms (e.g., at various locations 182, 282 and/or 284) for “beating heart surgeries” and procedures. They may also provide “sternum ease” using the vertical and horizontal barrel or crossbar 130 control of relationships 190 and 192 provided by the crossbar curves 102 and 104 when moving the moveable arm 140 away from or towards arm 120 along length 106. They may also provide a barrel or crossbar force of 50-50 power, that evens force distribution amongst and pivoting of blades 180. They may also provide a ratchet mechanism to overpower or override 50-50 opening forces, minimize damage. They may create an angle (e.g., created by the retractor) that reduces pressure on the first rib and the remaining sternum in comparison to the pressure near the abdomen. The triangular shape formed upon opening the retractor provides optimal visibility for the surgeon and helps prevent rib breakage at the rib cages or the surgeon making a full incision through the sternum. Since less opening near the sternum is required for heart access, the surgeon's maneuverability is significantly enhanced. Therefore, during full retraction, there is a force distribution that is lower near the top of the sternum, where the opening is smaller, and greater force is exerted lower down with a large opening. They may also provide a barrel shaped vector force at and parallel to the circumference of the separated front of the rib cage that reduces or prohibits the rib cage from folding outwards during heart surgeries, including bypass surgeries. The circumference of the front of the rib cage may want to fold out using other sternum spreaders that do not have the barrel shaped force provided by the arms and/or blades due to the technologies herein. This may be true for adults having cavity 207, top part 262 and/or bottom part 264 separated by between 5.5 and 7.5 inches. This may be true for children or pediatric patients having cavity 207, top part 262 and/or bottom part 264 separated by between 1 and 2 inches. This may be true without providing extreme stress on the circumference of the rib cage. This “barrel action” vector force minimizes broken ribs and neurological damage to the surrounding nerves during the sternum spreading, during the surgery and during the sternum closing.

It is considered that the sternum spreaders, crossbar 130, blade attachments 210 and 212, and/or attachment angle adjuster 296 herein can be used to spread other parts of a human patient such as to spread an incision between ribs, into the stomach, into the back, etc. It is considered that they can be used to spread similar parts of an animal.

In some cases, blades 180 may be flip up blades that can be tiled up vertically to be removed from being below the arms 120 and 140; and/or removed from being in cavity 207. It is considered that the spreader and attachment angle adjuster 296 can be configured to have top part 262 wider than bottom part 264 by extending length L1 using structure 298.

### CLOSING COMMENTS

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. With regard to flowcharts, additional and fewer steps may be taken, and the steps as shown may be combined or further refined to achieve the methods described herein. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

It is claimed:

1. A sternum spreader surgical instrument including a toothed crossbar having an arch wise curved and horizontal wise curved length, the spreader comprising:
a stationary arm having a first end and a second end;
a toothed cross bar having a first end and a second end, the toothed cross bar first end being secured to the first end of the stationary arm in a generally perpendicular relation and forming a generally L-shaped section;
a moveable arm having a first end and a second end, the first end of the moveable arm coupled to the toothed cross bar in a generally perpendicular relation;
the first end of the movable arm having a housing with a slotted opening which is attachable to the toothed cross bar and is laterally moveable along the length of the cross bar;
rack and pinion structure in the housing for moving the moveable arm along the length of the cross bar;
the stationary arm and the moveable arm being oriented generally perpendicular to the length of the cross bar;
each of the moveable arm and the stationary arm attached to a triangular shaped blade attachment having blades connected to them that are configured to engage the walls of a surgically cut opening on a patient, and the movable arm can be extended away from the stationary arm by rotating the rack and pinion structure to spread apart and hold open the walls of the opening thereby exposing a body cavity for allowing the surgeon to operate in the body cavity; and
the toothed crossbar having the arch wise curved and horizontal wise curved length such that the stationary arm and the moveable arm are capable of being: 1) spaced in both a generally arch wise non-parallel relationship with a bottom surface of the stationary arm forming an angle with a bottom surface of the moveable arm, and a generally horizontal wise, non-parallel relationship with a length of the stationary arm forming an angle with a length of the moveable arm, and 2) opened and closed in both the generally arch wise, non-parallel relationship, non-parallel relationships to each other by rotating the rack and pinion structure in the housing of the moveable arm.

2. The sternum spreader surgical instrument of claim 1, wherein the arch wise curve of the length has an arch distance ACH of between 0.25 to 3 inches at a middle of the crossbar length; and wherein the horizontal wise curve of the length has a horizontal curve distance CRV of between 0.25 and 3 inches at the middle of the crossbar length.

3. The sternum spreader surgical instrument of claim 1, wherein the stationary arm and the moveable arm are arch wise in a non-parallel relationship with each other such that a top and a bottom surface of the stationary arm and the moveable arms have an arch wise angle between them that increases as the movable arm is extended away from the stationary arm; and
wherein the stationary arm and the moveable arm are horizontal wise in a non-parallel relationship with each other such that a horizontal wise angle between a length of the stationary arm and a length s of the moveable arm increases as the movable arm is extended away from the stationary arm.

4. The sternum spreader surgical instrument of claim 1, wherein the triangular shaped blade attachments comprise:
a first and second triangular shaped blade attachment, each having a proximal fastener removably attached to the stationary and moveable arms and each having two distal blade mounts for mounting a first and second blade.

5. The sternum spreader surgical instrument of claim 4, wherein the fasteners swivel with respect to a tangent to a length between the first and second ends of the arms so the triangular shaped blade attachments swivel and an angle that changes between the length of the arms and the tangent.

6. The sternum spreader surgical instrument of claim 4, wherein the fasteners are removable from the arms so the blade attachments can be interchanged with other triangle attachment adjusters; and
further comprising multiple positions along the stationary arm and the moveable arm for locating the blade attachments.

7. The sternum spreader surgical instrument of claim 4, further comprising a first attachment angle adjuster attached to and between the moveable arm and an arm of the attachment extending from a location on the arm of the of the blade attachment to an angle adjuster mounting location, the adjuster having an extendable length rack and pinion structure to change the length and an angle between the arm of the of the blade attachment and the moveable arm.

8. The sternum spreader surgical instrument of claim 4, wherein the blade mounts swivel with respect to the blade attachment so an angle changes between a flat surface of the blade and a line connecting the two blade mounts; and
wherein the blade mounts are removable mounts so the blades can be interchanged with other blades.

9. The sternum spreader surgical instrument of claim 1, wherein the rack and pinion structure includes a circular gear having pinion teeth for engaging a linear gear having rack teeth of along a rear surface of the length of the cross bar that is away from the arms; and
wherein the crossbar teeth have one of: a wider distal end away from the cross bar than proximal end towards the cross bar, or a wider spacing between the distal ends than between the proximal ends.

10. The sternum spreader surgical instrument of claim 1, wherein the moveable arm and stationary arm each include an arm angle adjustor to change an angle between a proximal length of the arm and a distal length of the arm.

11. A chest spreader surgical instrument including a crossbar having an arch wise curved and horizontal wise curved length, the spreader comprising:
a stationary arm having a first end and a second end;
a cross bar having a first end and a second end, the cross bar first end being secured to the first end of the stationary arm in and forming a generally L-shaped section;
a moveable arm having a first end and a second end;
the first end of the movable arm having a housing with a slotted opening which is attachable to the cross bar and is laterally moveable along the length of the cross bar;
each of the moveable arm and the stationary arm having a triangular shaped blade attachment having blades connected to them and extending below the blade attachments such that the blades can engage the walls of a surgically cut opening on a patient, wherein the movable arm can be extended away from the stationary arm by rotating the rack and pinion structure to spread apart and hold open the walls of the opening thereby exposing a body cavity for allowing the surgeon to operate in the body cavity; and
the moveable arm and stationary arm capable of being: 1) spaced in with their first ends and their second ends separated by the blade attachments, and 2) opened and closed by rotating the rack and pinion structure in the housing of the moveable arm.

12. The chest spreader surgical instrument of claim 11, wherein the triangular shaped blade attachments comprise:
a first and second triangular shaped blade attachment, each having a proximal fastener removably attached to the stationary and moveable arms and each having two distal blade mounts for mounting a first and second blade.

13. The chest spreader surgical instrument of claim 12, wherein the fasteners swivel with respect to a tangent to a length between the first and second ends of the arms so the triangular shaped blade attachments swivel and an angle that changes between the length of the arms and the tangent.

14. The chest spreader surgical instrument of claim 12, wherein the fasteners are removable from the arms so the blade attachments can be interchanged with other triangle attachment adjuster; and
further comprising multiple positions along the stationary arm and the moveable arm for locating the blade attachments.

15. The chest spreader surgical instrument of claim 12, further comprising a first attachment angle adjuster attached to and between the moveable arm and an arm of the attachment extending from a location on the arm of the of the blade attachment to an angle adjuster mounting location, the adjuster having an extendable length rack and pinion structure to change the length and an angle between the arm of the of the blade attachment and the moveable arm.

16. The chest spreader surgical instrument of claim 12, wherein the blade mounts swivel with respect to the blade attachment so an angle changes between a flat surface of the blade and a line connecting the two blade mounts; and
wherein the blade mounts are removable mounts so the blades can be interchanged with other blades.

17. The chest spreader surgical instrument of claim 11, wherein the moveable arm and stationary arm each include an arm angle adjustor to change an angle between a proximal length of the arm and a distal length of the arm.

18. A method of opening and closing a sternum spreader surgical instrument including a toothed crossbar having an arch wise curved and horizontal wise curved length, the method comprising:
rotating a pinion of a rack and pinion structure to move a mobile arm towards a stationary arm, wherein rotating includes moving the mobile arm along a toothed crossbar having an arch wise curved and horizontal wise curved length so that the arms are in a parallel relationship; and
un-rotating the pinion of a rack and pinion structure to move the mobile arm away from the stationary arm, wherein un-rotating includes moving the mobile arm along the toothed crossbar so that the arms are in a non-touching non-parallel relationship.

19. The method of claim 18, wherein each of the mobile arm and the stationary arm are attached to a triangular blade attachment, and wherein each triangular blade attachment is attached to two blades.

20. A method for configuring a sternum spreader, the method comprising:
selecting two triangular blade attachments to attach to two arms of the spreader;
attaching the two triangular blade attachments to the spreader;
selecting an angle adjuster to attach between a spreader arm and an arm of a blade attachment;
attaching the angle adjuster between the spreader arm and the arm of a blade attachment;
rotating a pinion of a rack and pinion structure to move at least one opposing blade of each of the arms in a touching position; and
rotating the pinion of a rack and pinion structure to move the at least one opposing blade of each of the arms in a non-touching and non-parallel position.
